(19) United States
(12) Patent Application Publication
(10) Pub. No.: US 2010/0021398 A1
(43) Pub. Date: Jan. 28, 2010

(54) COMPOSITIONS AND METHODS FOR LIGHTENING SKIN AND PROTECTING SKIN FROM ULTRAVIOLET RADIATION WITH GLUTATHIONE

(76) Inventor: Keith K. Skinner, Aurora, CO (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/355,666

(22) Filed: Jan. 16, 2009

Related U.S. Application Data
(60) Provisional application No. 61/022,165, filed on Jan. 18, 2008.

(51) Int. Cl. A61K 8/18; A61Q 17/04; A61Q 19/02

(52) U.S. Cl. 424/59; 424/62

(57) ABSTRACT

A glutathione composition for lightening or protecting skin is disclosed together with a method for lightening skin or protecting the skin from the effects of ultraviolet radiation is accomplished by administering glutathione to the skin. The glutathione may be glutathione modified with at least one fatty acid or the glutathione molecule is acetylated/esterified. In instances where it is acetylated/esterified, it may optionally be associated with cyclodextrin.

### RELATED APPLICATIONS

This application incorporates by reference and claims the Paris Convention Priority of U.S. Provisional Patent Application Ser. No. 61/022,165, which was filed on Jan. 18, 2008.

### BACKGROUND

This disclosure relates to the lightening using modified glutathione. The glutathione is modified to make it more readily uptaken into the tissue and cells when applied topically. Such modifications include the addition of fatty acid groups, as well as protection of the glutathione molecules using acetylation and esterification. Additionally, cyclodextrin may be used to protect glutathione through the digestive tract in oral versions of the methods of the present disclosure.

Skin without significant dyschromia is an aesthetic goal of people worldwide. Current options for lightening skin have shown to have significant drawbacks that are well known to artisans.

The color of the skin is determined by the amount and type of melanin synthesized by melanocytes in the dermal tissue and the distribution pattern of that melanocyte in the surrounding keratinocytes.

Melanin forms through a series of oxidative reactions involving the amino acid tyrosine in the presence of the enzyme tyrosinase. Tyrosinase catalyses three different reactions in the biosynthetic pathway of eumelanin melanin in melanocytes:

Hydroxylation of tyrosine to l-DOPA;

l-DOPA to dopaquinone;

dopaquinone to melanin.

Phaeomelanin is converted to melanin through 5-S-cysteinyldopa.

### SUMMARY

A glutathione composition for lightening or protecting skin is disclosed together with a method for lightening skin or protecting the skin from the effects of ultraviolet radiation is accomplished by administering glutathione to the skin. The glutathione may be glutathione modified with at least one fatty acid or the glutathione molecule is acetylated/esterified. In instances where it is acetylated/esterified, it may optionally be associated with cyclodextrin.

According to a feature of the present disclosure, a method is disclosed comprising administering to a subject an effective amount of glutathione to lighten the skin of the organism or protect the organism from the effects of ultraviolet radiation. The may be glutathione modified with at least one fatty acid or the glutathione molecule is acetylated/esterified. In instances where it is acetylated/esterified, it may optionally be associated with cyclodextrin.

According to a feature of the present disclosure, a composition is disclosed comprising an effective amount of glutathione administered to a subject to lighten the skin of the patient or to protect the subject from the effects of exposure to ultraviolet radiation; and a pharmaceutically acceptable carrier. The may be glutathione modified with at least one fatty acid or the glutathione molecule is acetylated/esterified. In instances where it is acetylated/esterified, it may optionally be associated with cyclodextrin.

### DRAWINGS

The above-mentioned features and objects of the present disclosure will become more apparent with reference to the following description taken in conjunction with the accompanying drawings wherein like reference numerals denote like elements and in which:

FIG. 1 is a block diagram illustrating the effect of tyrosinase intraction with GSH (glutathione).

FIG. 2 is a flow diagram of a process of lightening skin using modified glutathione.

### DETAILED DESCRIPTION

These embodiments are described in sufficient detail to enable those skilled in the art to practice the invention, and it is to be understood that other embodiments may be utilized and that logical, mechanical, electrical, biological, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

U.S. Utility Patent Application Publication Nos. 2008/0027212 and 2008/0311093, are hereby incorporated by reference as if fully disclosed herein. U.S. Utility application Ser. No. 12/142,698, filed on Jun. 19, 2008 is hereby incorporated by reference as if fully disclosed herein.

The inhibition of tyrosinase is a viable method for lightening skin. Alternately, inhibition of either eumelanin or phaeomelanin may accomplish the same. The present inventor has discovered that administration of glutathione to skin may reverse the production of melanin and lighten the skin. Furthermore, administration of glutathione is useful for protecting the skin against the effects of ultraviolet radition exposure.

Skin lightening may be accomplished through the phaeomelanin pathway by reduction of 5-S-cysteinyldopa (5-S-CD). It has been proposed that that addition of cysteine to dopaquinone is the main source of 5-S-CD in human epidermal melanocytes. A mechanism for regulating dopaquinone levels during pigment formation or a defense mechanism against oxidative stress is through the synthesis of 5-S-CD.

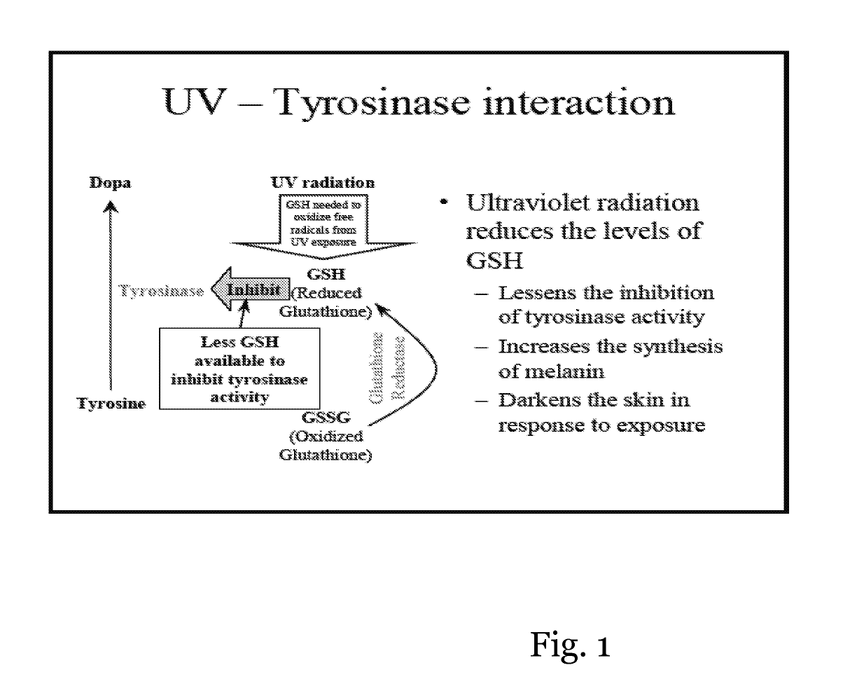

It has been discovered that when the level of glutathione is lowered, the level of 5-S-CD increases. For example, FIG. 1 illustrates the effect of ultraviolet light on the activity of tyrosinase, thereby causing the skin to darken via reduction of glutathione (GSH). Thus, when the level of glutathione is raised, 5-S-CD is lowered and the skin is lightened.

Moreover, protective anti-oxidant enzymes and intracellular glutathione is essential for cell health and survival. This was verified in experiments where mice were exposed to ultraviolet light B (UVB). The response of the skin and serum anti-oxidant enzymes like superoxide dismutase (SOD), catalase, glutathione peroxidase (GSH-Px) were examined as a result. The study found that lipid peroxides were increased at 3 and 24 hr after irradiation with UVB. However, the four reactive oxygen species (ROS) scavenging enzymes were generally decreased during the first 48 hr after exposure to UVB. Thus, after the UVB exposure free radicals are present in high concentrations above normal within three hours of UVB irradiation and the protective anti-oxidant enzymes were decreased for at least two days.

Consequently, a topical pharmaceutical having glutathione that is delivered intracellularly provides a novel treatment for exposure of skin to ultraviolet light. Optionally, the pharmaceutical could have other anti-oxidant enzymes as well that would prevent damage from the free radicals present after exposure to UVB.

Additionally, glutathione delivered intracellularly will inhibit tyrosinase, and even casual sun exposure will not reverse the skin lightening effect. Additionally, glutathione was shown to up-regulate the anti-oxidant enzymes. For example, SOD was increased 182%.

Thus, the compositions of the present disclosure, while useful for lightening the skin, also have a secondary effect of protecting the skin for UV exposure. According to embodiments, a composition having 0.4% glutathione were shown to provide the following benefits:

1. 182% increase of Mitochondrial SOD 2, which is above youthful cell levels.

2. An increase of Vimentin above youthful levels (140% increase).

3. Improves ICAM-1 (143%) to almost youthful levels.

Modification of organic molecules is an unpredictable art in which results may be different from expectations and methods of modifying organic molecules of necessity become very complex. The method of the present disclosure, however, is straightforward and yet yields effective forms of the molecules.

Glutathione has been shown to be active as a skin lightening agent. The inventors have discovered that modified glutathione is more readily bioavailable and propose methods of lightening skin using glutathione molecules that are modified with fatty acids or through acetylation and esterification, which aid in their uptake and bioavailability to cells over conventional methods.

Glutathione is a three amino acid peptide chain consisting of glycine-cysteine-glutamic acid. It is a potent agent for the purposes of skin lightening and for tissue exposed to UV radition. Thus, intracellular delivery of glutathione is highly desirable from the standpoint of health maintenance. However, as a peptide, glutathione is subject to the proteases of the digestive tract. Moreover, glutathione has a half-life of about a minute and a half in the blood stream.

Thus, to effectively deliver glutathione intracellularly, the glutathione molecules must be “protected” from the harsh environment of the digestive tract, or presented in a form for easy and efficient uptake through non-oral delivery mechanisms. The following methods for delivering glutathione rely on reversible modification of the glutathione molecules by fatty acids, or protection of glutathione via acetylation and esterification and optionally protection with cyclodextrin.

### I. Fatty Acid Modification of Glutathione

Accordingly to embodiments, glutathione may be modified by bonding fatty acids to active sites of the glutathione to increase the fat solubility, which helps the glutathione molecule in absorption through the skin as well as absorption through the cell membranes of cells. Consequently, modified glutathione is a more effective vehicle over traditional methods of glutathione delivery because of its increased ability to be absorbed when applied topically, as well as being absorbed by cells generally.

The present disclosure uses fatty acid modified agents, such as glutathione or other skin lightening agents, to make the agents more readily bioavailable and effectively lighten the skin. Adding fatty acids to agents further allows the skin to more easily and efficiently absorb the agent through skin. In effect, adding fatty acids to agents creates both a vehicle for delivery through lipid bilayers of cells and the skin, and a “time release” effect as the agent is not bioavailable until the lipid side chains of the modified agent are cleaved. Fatty acids are cleaved carbon by carbon. Thus, agents having longer fatty acids chains therefore take longer to become bioavailable than those having shorter fatty acid chains.

The present disclosure proposes a novel method of making agents more deliverable to tissue or cells for the purpose of skin lightening by adding fatty acids to active sites on the agents. The fatty acids are covalently bonded to one or more active sites of the agent. For example, the fatty acids may be bonded to the active sites of glutathione, such as the sulphydryl group, the amine groups, and the carboxyl groups.

Agents have an active site that can reversibly react with the carboxyl end of fatty acids; these may include NH₂, SH, and OH sites. Indeed, the sites are preferentially bound amino or any free binding site, then sulphydryl or any free binding site, and finally hydroxyl. As will readily be recognized by artisans, NH₂ sites are will be modified first due their positive charge.

However, modification of the hydroxyl active sites is advantageous because ether bonds form between the fatty acid and the agent. The ether bonds are more stable in biologic systems, which means that the cell takes longer to break down the fatty acid and expose the active site.

According to embodiments, agents that may be modified according to the present disclosure include, but are not limited to, glutathione and glutathione variants, and many other agents that have that are able to effect lightening of skin.

As well known to artisans, fatty acids comprise an aliphatic chain coupled to a carboxylic acid. According to embodiments, the carboxy end of fatty acids are reacted to the active sites of agents. The fatty acid-agent complex serves two purposes. First, the fatty acids reversibly block the active sites of the agents until the agent is delivered intracellularly. Second, the lipophilic aliphatic side chain or chains of the fatty acids allow the agent to more readily cross the cell membrane and penetrate skin, for example. Thus, by coupling skin lightening agents and fatty acids, a more potent method for the delivery of skin lightening agents and lightening skin is introduced.

According to embodiments, any fatty acid having two or more carbons in the aliphatic chain are suitable to be coupled to agents. The fatty acids may be saturated or unsaturated. According to embodiments, butanoic acid (C4:0), pantanoid acid (C5:0), hexanoic acid (C6:0), octanoic acid (C8:0), nananoic acid (C9:0), decanoic acid (C10:0), dodecanoic acid (C12:0), tetradecanoic acid (C14:0), hexadecanoic acid (C16:0), heptadecanoic acid (C17:0), octadecanoic acid (C18:0), icosanoic acid (C20:0), docosanoic acid (C22:0), tetracosanoic acid (C24:0), hexacosanoic acid (C26:0), heptacosanoic acid (C27:0), octacosonoic acid (C28:0), triacontanoic acid (C30:0), dotriacontanoic acid (C32:0), dotriacontanoic acid (C32:0), tritriacontanoic acid (C33:0), tetratriacontanoic acid (C34:0), or pentatriacontanoic acid (C35:0) are saturated fatty acids that are readily available and that are appropriate for use with the present disclosure. Fatty acids having more than 35 carbons and fatty acids having aliphatic chains of both an even and an odd number of carbons are equally applicable with the teachings of the present disclosure.

Similarly, unsaturated fatty acids having any number of double or triple bonds in both a -cis or -trans configurations are expressly contemplated. For example, myristoleic acid (C14:1), palmitoleic acid (C16:1), oleic acid (C18:1), linoleic acid (C18:2), α-linoleic acid (C18:3), arachidonic acid (C20:4), eicosapentaenoic acid (C20:4), eicosapentaenoic acid (C20:5), erucic Acid (C22:1), or docosahexaenoic acid (C22:6) are examples of common unsaturated fatty acids that may be coupled to agents according to the present disclosure. Other unsaturated fatty acids are expressly contemplated, as would be known to artisans.

Moreover, according to embodiments, the fatty acids of the present disclosure may be oils, such as olive oil, jojoba oil, sunflower oil, safflower oil, rapeseed oil, corn oil, soya oil, wheat germ oil, cottonseed oil, almond oil or oils of other nuts, palm oil, coconut oil, vegetable oil, butter, lard, as well as other oils comprising, at least in part, fatty acids. Obviously, where the agent is to be delivered intracellularly, the oil or fatty acid must be non-toxic.

According to embodiments, the oil selected my comprise oils known to be healthy, such as olive oil or omega-3 fatty acids. Use of such health-type oils may be of interest to the health food markets, etc. Moreover, according to embodiments the agents may comprise health food supplements to be sold as such or may be included as additives in containers of oil purchased, for example, at the grocery story for general cooking or spread purposes.

Once delivered intracellularly, enzymes within the cell cleave off the fatty acids, allowing the bioactive sites of the agent to become available. Cleaving of the fatty acids occurs carbon by carbon. Consequently, the longer the aliphatic chain of the fatty acid, the longer the agent will be protected by the fatty acid(s). Indeed, by using multiple oils having different size aliphatic chains, a time release-like product is created whereby the agents having the shorter aliphatic chains become bioavailable more quickly on average than those having longer aliphatic chains.

The process for protecting the agents with fatty acids is performed in aqueous solution using the fatty acid chloride of the fatty acids being used to modify. As will be seen, it may be performed in quantities of scale without appreciable modification in the core steps of the procedure. Initially, the agent of interest is dissolved into water. According to embodiments, the concentration of the agent in the water is increased to a maximum concentration.

After the agent to be modified is dissolved into water, the pH is raised to pH 12-13 with a base. According to embodiments, the base is an inorganic base, such as NaOH, which prevent undesirable side reactions. Throughout the modification process, the pH is kept in the range of pH 12-13 to drive the modification reaction. After the pH is raised to pH 12-13, the fatty acid chloride is added to drop-wise to the solution under agitating/stirring, together with additional base to maintain the desired pH. As the fatty acid chloride is added to each agent, the resulting product falls out of solution as a precipitate. According to similar embodiments, the solution need not have the pH raised before adding the fatty acid chloride and the base, whereby the pH will be raised as a matter of course during the reaction.

The precipitate is then harvested. Harvesting may occur simply by decanting the water, washing the precipitate with water at least once, and drying. The resultant dry precipitate is the agent coupled to one or more fatty acid molecules. The precipitate may then be added as an additive to other products such as vitamin tablets, lotion, etc. for delivery purposes. According to embodiments, fatty acid modified agent products by the disclosed process are expressly contemplated.

It will be understood by artisans that the methods of the instant disclosure may be performed on a large scale without appreciable changes to the principles disclosed by the exemplary protocol.

According to embodiments, the fatty acid modified agent products may be further modified, either before or after the process disclosed herein to provide further desirable characteristics. For example, agent molecules, such as glutathione, may be esterified prior to the process disclosed herein. Other similar modifications that are known in the art, such as acetylation with glutathione, are also possible and expressly contemplated, provided active sites on the agent are available for modification.

### II. Acetylation and Esterification Modification of Glutathione

According to an exemplary embodiment using glutathione, the agent, including glutathione, is stabilized by acetylation and esterification. For example, with glutathione, the three active sites of the glutathione molecule are modified, preventing enzymatic degradation in both the digestive tract and the blood stream. A benefit of the stabilization process described herein is that the number of hydrocarbons in the glutathione molecule is reversibly increased, which makes the glutathione molecule more hydrophobic and increases the ability of the molecule to be absorbed through the lipid rich cellular membrane and the skin.

Certain binding sites on glutathione molecules are altered to stabilize the glutathione molecule and prevent degradation prior to entering a cell. Moreover, the present disclosure discloses the use of “protector” molecules designed to effectively deliver through the digestive system modified glutathione molecules into the bloodstream.

Similarly, the hydrogen atom in the amino group may serve as a connection for alteration of the amino acid in a different manner, acetylation of the molecule into a different altered form, which altered form again alters the processes involved in metabolization of the molecule. Adding an acetyl group to either end will begin to render the molecule more likely to pass through early stages of metabolization and thus more likely to finally penetrate the cells.

The method and process of the present disclosure is not limited to amino acid molecules (nor even peptides). For explanatory purposes, however, it will be understood that the connections discussed above may be available in peptides and even in larger agents for lightening the skin, so the same effects are discussed in terms of virtually any peptide/protein of similar structure or other organic molecules having carboxyl, sulphydryl, amine groups, or other active groups subject to acetylation or esterification.

A process for acetylating and esterifying organic molecules is hereby disclosed using glutathione for example. According to embodiments, an acetylation reagent is prepared using 20 μL acetic anhydride and 60 μL of an alcohol or other suitable agent. The alcohol or suitable agent is methanol, ethanol, dimethyl formamide (DMF), and combinations thereof, as well as other suitable alcohols, according to embodiments. Acetylation using methanol and ethanol respectively may lead to the formation of methyl esters and ethyl esters. In proteins, glutathione for example, using DMF may lead to acetylation of either the N-terminal end of the molecule or the N-terminal end and the cysteine terminus.

Up to 1 nmol of an agent, such as glutathione, is reconstituted in 20 μL of 50 mM of ammonium bicarbonate. According to embodiments, the process may be provided to protect many agents, including from the group consisting of: glutathione and others agents for lightening the skin. Similarly, other organic molecules having active sites and the need to be protected in the digestive tracts and blood streams are similarly contemplated including vitamins, minerals, agents, enzymes, proteins, etc.

After the organic molecule is reconstituted, 50 μL of the acetylation reagent and 20 μL of the agent solution are combined and allowed to stand at a first temperature for an hour.

Thereafter, the degree of acetylation is determined. According to an embodiment, the final degree of acetylation depends on the specific experimental conditions. The reaction is thermogenic and thus allows reasonably fine control over the degree of alteration of the organic molecule, in particular, the number of acetyl and ester groups added. According to embodiments, modification occurs at one or all active sites of the exemplary glutathione molecule at the amino terminal end, carboxyl terminal end, the sulphydryl group of the cysteine, and the carboxyl side group of the glutamic acid.

According to embodiments, glutathione may be suspended in an ethanol solvent. Naturally, the carboxyl groups will be esterified by the ethanol. When acetic anhydride is added, acetylation of the amino group and sulphydryl group will occur. Steric hindrance will become a factor as an increased number of the functional groups are either esterified or acetylated. Accordingly, the final result will comprise a solution having glutathione molecules with a varying degree of modification to the functional groups. Some glutathione molecules will have all 4 active sites modified, some with 3 of four, and so forth. Depending on the experimental conditions the acetyl to ester ratio is adjusted. For example, by lowering the pH of the solvent, a higher degree of esterification is observed.

According to embodiments, agitation (with bubbling nitrogen or mechanical stirring) for 24 hours produces an amino acetylated product. If stirring is carried out for 72 hours, a racemic mixture of amino and sulphydryl acetylation occurs in a mixture of roughly equal parts. Addition of acetic anhydride in a 10 fold ratio (molar) will shift the degree of acetylation from the 50/50 ratio to approximately 20 parts amino acetylation and 80 parts sulphydryl acetylation, in glutathione for example.

Temperature variations may also be used to alter the final form of the product. For example, glutathione at 10° C. above room temperature in an alcohol solvent reduces steric hindrance thus allowing both sides of the molecule to acetylate equally; where DMF is the solvent, the result is a 50/50 mix at 100% acetylation. Similarly, variation in the heating and mixing times, produces varying desirable results that may be determined without undue experimentation. Excessive heat demonstrates one possible disadvantage for glutathione and other sulphydryl containing organic molecules, however, which is bonding at the sulphydryl groups into dimers.

When temperature is reduced but agitation and heating time increases to 5 days (120 hours), thorough acetylation of the sulphydryl group of cysteine is accomplished.

The final product may be lyophilized or otherwise dried for later use in therapeutic products. As a final product, glutathione molecules that are acetylated and esterified at more sites are preferable because (1) acetylation and esterification protect the glutathione molecule as it is in route to a cellular target and (2) the increased molecular weight increases hydrophobicity and makes the molecule more readily absorbed through the skin and cellular membranes.

Accordingly, disclosed herein is a method for delivering a modified skin lightening agent into a cell when administered orally or topically. Although the principles disclosed herein are applicable to many molecules as will be known and understood by artisans, glutathione is again used by way of illustration.

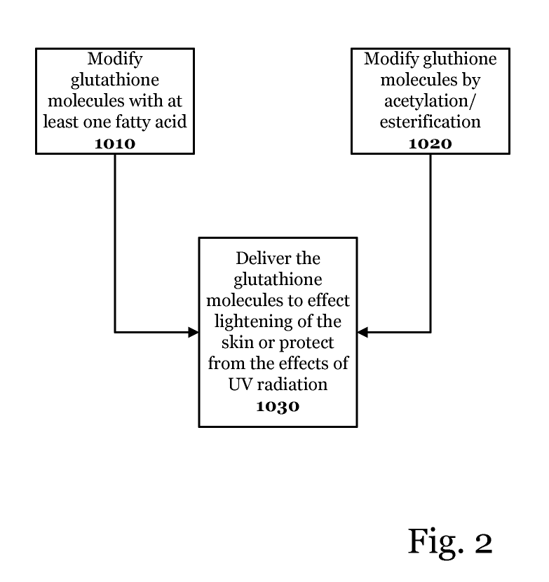

As illustrated in FIG. 2, a method of the present disclosure is illustrated. According to the method, an agent is modified with a fatty acid, as disclosed herein, in operation 1010 or by acetylation/esterification in operation 1020. Thereafter, the modified agent is delivered by topical or oral administration in operation 1030.

According to embodiments, glutathione is modified by acetylating, esterifying, or modifying by a fatty acid the functional groups of the glutathione molecule, as described herein, for example. Such modification of the functional groups of glutathione prevents enzymes from degrading glutathione in the bloodstream.

Thereafter, according to embodiments where the glutathione is administered orally, the organic molecule is further protected to allow delivery the molecule through the digestive tract to the large intestine. According to embodiments, each glutathione molecule is placed into a cyclodextrin “bucket.” Accordingly, the amino end of the glutathione molecule is held in the cycledextrin ring via eletrostacic forces (as the inner portion of the cyclodextrin ring in more hydrophobic). As the ring resides on the amino terminal end of the glutathione molecule, proteases secreted in the digestive tract are unable to degrade the peptide bonds. Once in the large intestine, cyclodextrin is naturally degraded and the glutathione is absorbed through the wall of the large intestine into the blood stream.

Complexation of the organic molecule with cyclodextrin is accomplished by suspending the organic molecule to a 60% concentration of lab water (purified and filtered to 0.2 micron, millipore) in operation 2000. This equates to 600 mg/ml by weight. Alpha, beta, or gamma cyclodextrin is added to the organic molecule/water mixture. The mixture is slow stirred for 24 hours until a gel consistency is formed. The gel formation is indicative of complexation of the organic molecule with cyclodextrin. Large vessels are chosen, as this complex swells overnight at a ratio of 1 ml increase in total volume per 1 mg of cyclodextrin used. This gel is then re-diluted with an additional 10% water to give a slurry. This consistency is kept at room temperature and prepped for spray drying, lyophillization, or vacuum shelf drying. For large scale production, spray drying is appropriate.

According to embodiments, other agents may serve a similar function to that of cyclodextrin, namely: Eudrait RS 100 microparticles. Additionally, according to embodiments, Gliadin may be used to increase uptake of the glutathione molecules from the digestive tract into the bloodstream. A number of compounds are known which act like coatings or containers for the molecules. This group includes, but is not limited to, the use of cyclodextrin, microspheres, nanoparticles of the proper types and properties, and similar compounds, coatings, and containers now known or later discovered.

Other compounds may also increase delivery and penetration of the agents. Chitosan is known to bind to the mucosal layer of the intestinal wall, thus preventing the layer from binding to the delivered molecule and thus allowing the delivered molecule to have a better chance of success in penetration. The action of Gliadin and methylcellulose is analogous to cyclodextrin, as these compounds bind to branches of the organic molecule and thus protect that branch from enzymatic attack or the like.

Liposomes may provide the agents of the present invention with an additional layer of fat around the molecule, thus further increasing the lipophilic tendencies of the molecule, again making skin and cell membrane penetration more likely. In general, enteric coatings of any type, known or later developed, may be used to prevent or reduce enzymatic attack in the digestive tract.

Laboratory tests on this method have been carried out by applying cyclodextrins to acetylated glutathione esters in order to further increase efficiency of delivery and penetration. Wacker Chemical Co. of Adrian, Mich. provides a product named “Cavamax W8” (trademark of Wacker Chemical Co., not related to the present applicants) which brand of cyclodextrin has been used in testing.

Once in the bloodstream, the presence of the acetyl and ester groups prevent degradation of the glutathione molecule in the bloodstream. For example, because the half-life of glutathione in the blood is relatively short—around 120 seconds—modification of the functional groups extends the half-life considerably as it travels to a cell for uptake by the cell. Because the cellular membrane is hydrophobic, the modification makes the glutathione molecule more hydrophobic, which helps the glutathione molecule pass through the cellular membrane.

Referring still to the exemplary embodiment, when such a glutathione molecule is provided which has been more heavily altered with the addition of two, three or even four acetyl groups (a total acetyl group weight gain of +42 over the preexisting weight of the organic molecule per acetyl group added) it becomes fat soluble, or with a fatty acid, and thus less prone to linger outside of the cell, as water soluble peptides/proteins such as glutathione in their natural form are not conveyed into the cell efficiently. This further increases the ability of the molecule to form a useful cream, oil or emulsion, depending on form, thus increasing its suitability for dermal application. As glutathione has been shown to be of benefit in skin cell rejuvenation and thus wrinkle reduction, such an application is very desirable for such purposes, as well as skin lightening.

Once inside the cell, the acetyl, ester, and fatty acid groups are naturally cleaved. Thus, active glutathione molecules are delivered inside of a cell without degradation in the digestive tract or bloodstream.

According to embodiments, glutathione forms having only one or two added acetyl groups may be more useful in powder form, as one example. Oral application allows use for other purposes by the metabolism of the patient, yet the addition of the acetyl or fatty acid groups still allows the peptide to penetrate the cells with much greater efficiency than would otherwise be the case.

Additional methods for increasing the efficacy of the molecules, in particular by increasing the efficiency of delivery and penetration, are also available for use with the method of the invention.

Methods of delivery of the peptides to be delivered may thus be varied by adjusting the penetration aids discussed and by adjusting the lipophilic/hydrophilic balance of the molecule. As a result, sublingual delivery, oral delivery, cutaneous delivery, subcutaneous delivery, direct bolus delivery, IV drip delivery, and other methods are contemplated.

Another method of delivery is to use a small strip or other body of material which may dissolve in the mouth of the patient. This allows a solid form of the therapy but has the advantages of sublingual or mucosal delivery. In particular, the enzymes of human saliva are only capable of dissolving carbohydrates, not of breaking down proteins or peptides or in fact most types of organic molecules. This means that the first three barriers discussed herein, the enzymatic attack in the stomach, the mucous barrier of the intestinal wall, and the intestinal wall itself, may all be entirely circumvented. However, the patient convenience of having a portable, solid form, exact dosing mechanism is preserved.

Another method of delivery is the transdermal administration of the agents, for effective use not just by the skin or in a topical fashion but actually for use systemically or by other organs of the body.

### Pharmaceutical Compositions

The instant disclosure also provides pharmaceutical compositions. In some implementations, the pharmaceutical compositions comprise agents, acetylated/esterified glutathione, acetylated/esterified glutathione complexed with cyclodextrin, or fatty acid modified glutathione, which in such pharmaceutical compositions form the “active compound” or “agent.” According to implementations, the pharmaceutical compositions are administered to a subject to in need of anti-bacterial therapy, including gram-negative bacteria. According to other implementations, the pharmaceutical compositions are administered to a subject in need of lightening of the skin or prevention of damage from UV radiation.

In addition to active compound, the pharmaceutical compositions preferably comprise at least one pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Subject as used herein refers to humans and non-human primates (e.g., guerilla, macaque, marmoset), livestock animals (e.g., sheep, cow, horse, donkey, pig), companion animals (e.g., dog, cat), laboratory test animals (e.g., mouse, rabbit, rat, guinea pig, hamster), captive wild animals (e.g., fox, deer) and any other organisms who can benefit from the agents of the present disclosure. There is no limitation on the type of animal that could benefit from the presently described agents. Human subjects are expressly contemplated. A subject regardless of whether it is a human or non-human organism may be referred to as a patient, individual, animal, host, or recipient.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a container or dispenser which contains a suitable propellant, e.g., a gas such as carbon dioxide, or a nebulizer. Other delivery methods and devices common in the art, including mechanically actuated atomizing-like devices are expressly contemplated.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For epidermal, dermal, or transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one implementation, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in subjects. The dosage of such compounds lies preferably within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in subjects. Levels in plasma can be measured, for example, by high performance liquid chromatography.

As defined herein, a therapeutically effective amount of an active compound of the disclosure may range, for examples, from about 0.001 to 30 mg/kg body weight, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. Without limitation, the active compound can be administered between one time per week and three or more times per day, for between about 1 to 10 weeks, for example between 2 to 8 weeks, between about 3 to 7 weeks, or for about 4, 5, or 6 weeks. The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a pharmaceutical composition of the disclosure can include a single treatment or, preferably, can include a series of treatments.

### Examples

### Example 1

The methods of the present disclosure may be used to make a modified glutathione molecule. Glutathione is a potent agent having three primary active sites: the carboxy and amino terminal ends of the peptide sequence, as well as the sulphydryl residue of the cysteine amino acid. According to embodiments, unmodified glutathione or previously esterified glutathione is dissolved into water. Sodium hydroxide is added to bring the pH of the solution to pH 12-13. The solution is constantly stirred or agitated while a solution containing a palmitic acid chloride is added drop-wise into the water-glutathione solution. Concurrently, additional sodium hydroxide is added to the solution to maintain the pH at between 12-13. Under these conditions, the palmitic acid reacts with the active sites of the glutathione and the modified glutathione falls out of solution. Artisans will recognize that the palmitic acids reacts first with the amino residue, followed by the sulphyrdel residual, and then finally the carboxyl residue.

The reaction is propagated until an efficient yield of modified glutathione is precipitated. Thereafter, the water from the glutathione solution is decanted, whereby all unreacted fatty acid and glutathione is removed. The precipitate is washed one or more times to remove residual unreacted fatty acid and glutathione, as well as to decrease the pH to physiologically acceptable levels. After washing, the precipitate is dried.

Thereafter, the precipitate may added to lotions or topically applied vitamins, for example. The modified palmitated glutathione is a better deliverable because the fatty acid makes the glutathione molecule more readily absorbed through the skin or cell membrane permeable. Moreover, the palmitate protects the glutathione in transit until the fatty acid is fully cleaved from the glutathione molecule.

### Example 2

Similarly, the procedure of EXAMPLE 1 is duplicated. However, rather than using palmitic acid as the fatty acid, olive or jojoba oil chlorides are added as the fatty acid chloride. Artisans will readily recognize and understand the process of making the olive or jojoba oil chloride. The resulting olive oil-glutathione or jojoba oil-glutathione may then be marketed in health food stores as agent enhanced oils to be used in cooking or other desirable applications.

### Example 3

Oils that have multiple fatty acids, each having different sized aliphatic chains may be used to create “time-release” agents. Shorter aliphatic chains are cleaved more quickly to expose the active site of agents, while the longer aliphatic chains are protected for longer. Thus, the net effect is an extended delivery time for the modified agents.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
administering to a subject an effective amount of modified glutathione to lighten the skin of the organism or protect the organism from the effects of ultraviolet radiation;
wherein the glutathione is modified with at least one fatty acid or the glutathione molecule is acetylated/esterified.

2. The method of claim 1, wherein the fatty acid comprises at least palmitoleic acid.

3. The method of claim 1, wherein the fatty acid comprises at least one oil.

4. The method of claim 3, wherein the oil is selected from the group consisting of: jojoba oil, olive oil, or sunflower oil.

5. The method of claim 1, wherein the glutathione molecule is associated with at least one cyclodextrin molecule.

6. The method of claim 1, wherein the pharmaceutically acceptable carrier is a skin cream or lotion.

7. The method of claim 1, further comprising at least one antioxidant for reducing free radicals in the skin.

8. A composition comprising:
an effective amount of modified glutathione administered to a subject to lighten the skin of the patient or to protect the subject from the effects of exposure to ultraviolet radiation; and
a pharmaceutically acceptable carrier.
wherein the glutathione is modified with at least one fatty acid or the glutathione molecule is acetylated/esterified.

9. The composition of claim 8, wherein the fatty acid comprises at least palmitoleic acid.

10. The composition of claim 8, wherein the fatty acid comprises at least one oil.

11. The composition of claim 10, wherein the oil is selected from the group consisting of: jojoba oil, olive oil, or sunflower oil.

12. The composition of claim 8, wherein the glutathione molecule is associated with at least one cyclodextrin molecule.

13. The composition of claim 8, wherein the pharmaceutically acceptable carrier is a skin cream or lotion.

14. The composition of claim 8, further comprising at least one antioxidant for reducing free radicals in the skin.
